(19) United States
(12) Patent Application Publication
(10) Pub. No.: US 2026/0354991 A1
(43) Pub. Date: Nov. 12, 2026

(54) METHODS AND SYSTEMS FOR PROSTHESIS CONTROL

(71) Applicant: PHANTOM NEURO INC., Austin, TX (US)

(72) Inventors: Connor Guthman Glass, Austin, TX (US); Aurélien Philippe Bouvier, Woodside, CA (US); Erik Kolbjorn Lloyd, Dundas (CA)

(21) Appl. No.: 19/140,117

(22) PCT Filed: Dec. 21, 2023
(86) PCT No.: PCT/US2023/085385

§ 371 Date: Jun. 17, 2025

Related U.S. Application Data
(60) Provisional application No. 63/434,808, filed on Dec. 22, 2022.

(51) Int. Cl. A61F 2/72; A61B 5/00; A61B 5/11; A61B 5/296; A61B 5/397

(52) U.S. Cl. CPC A61F 2/72; A61B 5/0031; A61B 5/11; A61B 5/296; A61B 5/397; A61B 5/6861; A61B 5/7267; A61B 2560/0219

(57) ABSTRACT

Provided herein are implantable devices including: an implantable substrate including a sensor configured to wirelessly detect and wirelessly transmit electromyography (EMG) signals generated by one or more muscles of a subject, and a processor that is operatively coupled with the implantable substrate and configured to receive and transmit the EMG signals. The implantable substrate is configured to be implanted in the subject and in contact with the one or more muscles of the subject, and the implantable substrate may include one or more reference electrodes.

### TECHNICAL FIELD

This disclosure relates to implantable systems, implantable devices, and related methods of controlling a prosthesis.

### BACKGROUND

There are currently hundreds of millions of individuals worldwide with mobility impairments resulting from aging and/or physical disabilities. Robotic limb prostheses, active orthotics, and exoskeletons can help replace and/or augment the motor function of amputated or impaired biological limbs and allow users to perform daily activities that require the use of motorized orthopedic technologies, including those described in WO 2021/242775 A1, which is incorporated by reference in its entirety. However, the control of these wearable robotic devices is extremely difficult and often considered one of the leading challenges to real-world deployment. Thus, improvements in control of wearable robotic devices is continually sought.

### SUMMARY

In general, this disclosure relates to implantable systems, implantable devices, and related methods. Such implantable devices can be used for the wireless detection and transmission of EMG signals generated by one or more muscles of a subject.

In one aspect, this disclosure is directed to an implantable device, comprising: an implantable substrate comprising a sensor configured to wirelessly detect and wirelessly transmit an electromyography (EMG) signal generated by one or more muscles of a subject; and a processor operatively coupled with the implantable substrate and configured to receive and transmit the EMG signal, wherein the implantable substrate is configured to be implanted in the subject and in contact with the one or more muscles of the subject, and wherein the implantable substrate comprises one or more reference electrodes.

In some embodiments, the sensor is an EMG sensor.

In some embodiments, the implantable device further comprises a motion sensor.

In some embodiments, the EMG sensor and the motion sensor are configured to be inductively powered by an external power source.

In some embodiments, the implantable device further comprises a second EMG sensor configured to detect and transmit the EMG signal generated by a second muscle of the one or more muscles of the subject, wherein the sensor is a first sensor configured to detect and transmit the EMG signal generated by a first muscle of the one or more muscles of the subject.

In some embodiments, each of the first muscle and the second muscle is a skeletal muscle or a portion thereof.

In some embodiments, the implantable device is coated with or encapsulated within a biocompatible material and/or a bioinert material.

In some embodiments, the biocompatible and/or bioinert materials comprise silicone.

In some embodiments, the biocompatible and/or bioinert materials comprises at least one of a silicone material, a ceramic material, an epoxy material, a fabric, a cellular scaffold, an acellular scaffold, a plastic material, a metallic material, a carbon derivative, a synthetic biological tissue, a polymer, a meshed material.

In some embodiments, the processor is configured to wirelessly receive the EMG signal from the sensor.

In some embodiments, the processor is configured to wirelessly transmit the EMG signal to a wearable device.

In some embodiments, the wearable device comprises a self-contained battery; and a power transmitter configured to wirelessly transmit energy to the implantable device via an inductive magnetic field.

In some embodiments, the self-contained battery is rechargeable.

In some embodiments, the wearable device further comprises a power receiver.

In some embodiments, the power transmitter further comprises an amplifier configured to amplify a magnetic signal.

In some embodiments, the processor is a first processor, and wherein the wearable device comprises a second processor configured to: i) wirelessly receive the EMG signal from the first processor, and ii) wirelessly transmit the EMG signal to a computing device.

In some embodiments, the second processor receives the EMG signal from the first processor via a near field magnetic inductive link.

In some embodiments, the second processor transmits the EMG signal to the computing device via a short range communications link.

In some embodiments, the wearable device further comprises a decoder configured to wirelessly receive and decode the EMG signal.

In some embodiments, the sensor comprises one or more electrodes embedded within the implantable substrate.

In some embodiments, the implantable substrate has a first surface and a second surface on the opposite side of the first surface, and wherein the sensor is embedded within the first surface and the one or more reference electrodes are embedded within the second surface.

In some embodiments, the one or more electrodes comprise sensing electrodes.

In some embodiments, the sensor comprises two or more electrodes that are spaced equidistantly from a center of each of the two or more electrodes.

In some embodiments, the implantable substrate is configured to wrap around a circumference of the one or more muscles of the subject when implanted.

In some embodiments, the implantable substrate is a flat, elongated strip.

In some embodiments, the implantable substrate is flexible and configured to wrap around the one or more muscles of the subject.

In some embodiments, the implantable device further comprises a case connected to a connector of the implantable substrate, the case configured to house one or more electronic components.

In some embodiments, the case is composed of a biocompatible material and/or a bioinert material.

In another aspect, this disclosure is directed to an implantable system, comprising: one or more implantable devices comprising: an implantable substrate comprising a sensor configured to detect and transmit an electromyography (EMG) signal generated by a muscle of a subject, wherein the implantable substrate is in contact with the muscle of the subject; and a first processor operatively coupled with the implantable substrate and configured to: i) wirelessly receive the EMG signal from the sensor, and ii) wirelessly transmit the EMG signal; and one or more wearable devices configured to be attached to the subject, the one or more wearable devices comprising: a second processor configured to: i) wirelessly receive the EMG signal from the first processor, and ii) wirelessly transmit the EMG signal to a computing device; a self-contained battery; and a power transmitter configured to wirelessly transmit energy to the one or more implantable devices via an inductive magnetic field.

In yet another aspect, this disclosure is directed to a method of controlling a prosthesis comprising: detecting the EMG signal from the subject using any of the implantable devices of the disclosure; wirelessly transmitting the EMG signal from the implantable device to the wearable device; wirelessly transmitting the EMG signal from the wearable device to an external processing unit; processing the EMG signal using one or more machine learning classifiers; and based on the processing, generating a control output for the prosthesis.

In some embodiments, the control output comprises one or more of a continuous joint angle, a movement, or a discrete gesture.

In some embodiments, the one or more machine learning classifiers are one or more of a discrete classifier or a continuous classifier.

In some embodiments, the discrete classifier is a logistic regression classifier.

In some embodiments, the one or more machine learning classifiers are trained machine learning classifiers.

In some embodiments, the method further comprises training the machine learning classifier, wherein training the machine learning classifier comprises: receiving, via a processor, sensor data comprising one or more of the EMG signal or a motion signal corresponding to a movement of the subject; and training the machine learning classifier, via the processor, to generate one or more hyperparameters configured to be stored on the processor and configured to control the prosthesis.

Currently available robotic prostheses, active orthotics, and exoskeletons lack a means of voluntary active control that easily translates to improved quality of life during real-world utilization. Consequently, individuals with physical disabilities, such as amputees, and elderly individuals using these prostheses or exoskeletons need to rely on visual cues, mode toggling, physical manipulation, and inconvenient compensatory movements to harness the various functions that these solutions offer. The end-result is a burdensome and unintuitive process that provides minimal net benefit to the user, therefore leading to greater than 50% abandonment of such advanced robotic solutions.

Some embodiments of the devices, systems, and methods described herein may provide one or more of the following advantages. Some embodiments described herein may provide enhanced control of robotic prosthetic hands, elbows, shoulders, feet, ankles, knees, hips, and various combinations of such, as compared to currently available prosthetic devices and systems. Furthermore, in some embodiments, the systems, devices, and methods of the disclosure may provide enhanced control of robotic orthotics for both upper and lower limb functional reconstitution, as compared to currently available robotic orthotics. Furthermore, the systems, devices, and methods of the disclosure may provide enhanced control of robotic upper and lower limb exoskeletons for functional reconstitution and augmentation, as compared to currently available exoskeletons. Furthermore, the systems, devices, and methods of the disclosure may provide enhanced control of exosuits, as compared to currently available exosuits, and may provide enhanced control of future embodiments of robotic function augmenting systems. Lastly, the systems, devices, and methods of the disclosure may provide various non-orthopedic directed capabilities (e.g., nonverbal communication-to-speech, internet of things (IoT) control, enhanced augmented and virtual reality interactions, etc.)

The details of one or more embodiments of the invention are set forth in the accompanying drawings and the description below. Other aspects, features, and advantages of the invention will be apparent from the description, the drawings, and the claims.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated. Further, the term “about,” when used in connection with a referenced numeric value, is intended to include the referenced numeric value plus or minus up to 10% of that referenced numeric value, including increments therein. For example, the language “about 50” covers the range of 45 to 55.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a,” “an,” and “the” include plural references unless the context clearly dictates otherwise. For example, the term “a signal” includes one or more signals. “A and/or B” is used herein to include all of the following alternatives: “A,” “B,” “A or B,” and “A and B.”

As used herein, the term “subject,” “user,” or “patient” includes humans and mammals (e.g., mice, rats, pigs, cats, dogs, and horses). Typical subjects to which the implantable device of the present disclosure may be implanted in may include mammals, particularly primates, especially humans. For veterinary applications, suitable subjects may include, for example, livestock such as cattle, sheep, goats, cows, swine, and the like; poultry such as chickens, ducks, geese, turkeys, and the like; and domesticated animals, particularly pets such as dogs and cats. For diagnostic, therapeutic, and/or research applications, suitable subjects may include mammals, such as humans, rodents (e.g., mice, rats, hamsters), rabbits, primates, and swine such as inbred pigs and the like. The subject may be an amputee. The subject may have a muscle impairment.

As used herein, the term “bioinert material” refers to any material that once placed in vivo has minimal interaction with its surrounding tissue. Exemplary bioinert materials include, but are not limited to, silicone derivatives, ceramics, epoxies, fabrics, cellular scaffolds, acellular scaffolds, plastics, metals, synthetic biological tissues, bioabsorbable substrates, and meshes.

As used herein, the term “biocompatible material” refers to any material that does not deteriorate appreciably and does not induce a significant immune response or deleterious tissue reaction, e.g., toxic reaction or significant irritation, over time when implanted into or placed adjacent to the biological tissue of a subject, or induce blood clotting or coagulation when it comes in contact with blood. Exemplary biocompatible materials include, but are not limited to, silicone, ceramics, epoxies, fabrics, cellular scaffolds, acellular scaffolds, plastics, metals, synthetic biological tissues, plastics, polymers (such as polyimide) and meshes.

Unless otherwise defined, all technical and scientific terms used herein have the same meaning as commonly understood by one of ordinary skill in the art to which this specification pertains. Although methods and materials similar or equivalent to those described herein can be used to practice the invention, suitable methods and materials are described herein. In addition, the materials, methods and examples are illustrative only and not intended to be limiting.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of an implantable system, as described herein.

FIG. 2 is a perspective view of an example implantable device.

FIG. 3A is a perspective view of an implantable substrate of the implantable device of FIG. 2.

FIG. 3B is an enlarged view of the exposed portion of an electrode of the implantable substrate of FIG. 3A.

FIG. 4A is an enlarged, perspective view of an electronic module of the implantable device of FIG. 2.

FIG. 4B is an enlarged, side view of an example connector of the electronic module of FIG. 4A located at a first position.

FIG. 4C is an enlarged, side view of an example connector of the electronic module of FIG. 4A located at a second position.

FIG. 5A is a perspective view of an example implantable device.

FIG. 5B is a perspective view of an example implantable substrate.

FIG. 5C is a perspective view of an example feedthrough connector.

FIG. 5D is a perspective view of an example feedthrough connector.

FIG. 6A is a perspective view of a wearable device.

FIG. 6B is a top view of the wearable device of FIG. 6A.

FIG. 6C is a perspective, cross-sectional view of the wearable device of FIG. 6A.

FIG. 7A is a perspective view of a circular wearable device.

FIG. 7B is a perspective view of a rectangular wearable device.

FIG. 8 is a schematic diagram depicting examples of angular misalignment, lateral misalignment, and depth of the implantable substrate with respect to a wearable device.

FIG. 9 is a schematic diagram showing an example of an implantable system, as described herein.

FIG. 10A is a top view of an example implantable device of the implantable system of FIG. 9.

FIG. 10B is a bottom view of the example implantable device of FIG. 10A.

### DETAILED DESCRIPTION

### Implantable System

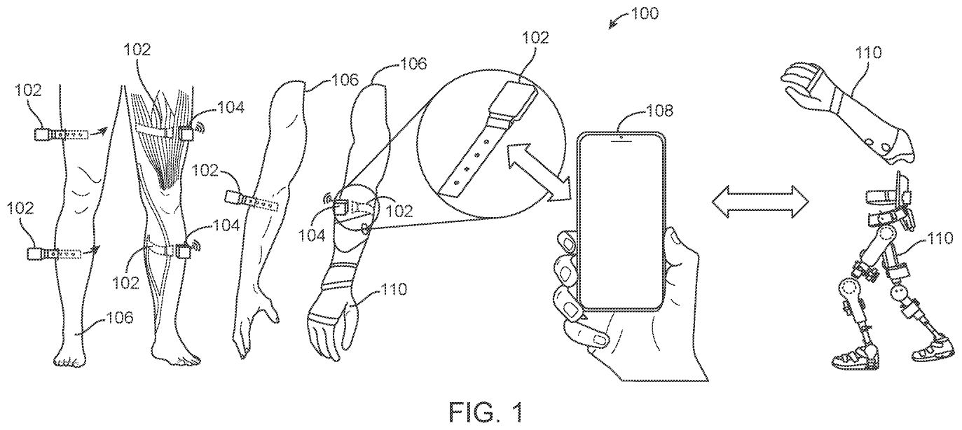

FIG. 1 is a schematic diagram showing an example of an implantable system 100 that can be used to control an orthopedic device (e.g., a prosthesis and/or an exoskeleton) via the detection of EMG signals generated by one or more muscles of a subject 106. The implantable system 100 includes one or more implantable devices 102 that can be implanted in a pocket formed within the subcutaneous or subadipose or subfascial anatomical planes in a subject 106, a wearable device 104 that can be removably secured to a limb of the subject 106, an external processing unit (EPU) 108, and a peripheral device 110.

The implantable device 102 includes one or more EMG sensors that can detect EMG signals generated by one or more muscles of the subject 106. The wearable device 104 can wirelessly connect to, power, and recharge the implantable device 102 when placed on a skin surface of the subject, near the vicinity where the electronic module 114 of the implantable device 102 is located. In this manner, the wearable device 104 can wirelessly transmit data (e.g., EMG data, motion data, a configuration parameter, a status parameter, and/or other types of sensor data) to and from the implantable device 102 via a wireless induction link, or other type of wireless communication system. In some embodiments, the wireless communication system includes, but is not limited to, a galvanic communication system, a capacitive communication system, a radio frequency (RF) communication system, an inductive communication system, an ultrasound communication system, an optical communication system, and a molecular communication system. From the wearable device 104, the data is configured to be transmitted either via wireless link, or a hardwired link to the EPU 108. The EPU 108 can be a smartphone or other portable processing unit. The EPU 108 can be a part of the wearable device 104 or the peripheral device 110. In some embodiments, the EPU 108 may be a central processing unit (CPU), a graphics processing unit (GPU), neural processing unit (NPU) or a neuromorphic processor, or any other specialized processor for running machine learning algorithms. On the EPU 108, the data is received from the wearable device 104 where it is processed and analyzed by one or more algorithms (e.g., a machine learning classifier or model, time-series signal processing).

As shown in FIG. 1, the implantable system can include two or more implantable substrate 102 that can work as a cohesive system where the two or more implantable substrate 102 can wirelessly connect to, be powered by, and be recharged by two or more wearable devices 104. In this manner, the wearable devices 104 can wirelessly transmit data (e.g., EMG data, motion data, and/or other types of sensor data) to and from the two or more implantable substrates 102 via a wireless induction link, or other type of wireless communication system. In some embodiments, all of the wearable devices 104 of the system can wirelessly transmit data to and from all of the implantable substrates 102 of the system. In some embodiments, a specific wearable device 104 in an implantable system 100 including two or more wearable devices 104 can be configured to wirelessly transmit data only to and from a specific implantable device 102 from all of the implantable devices 102 of the implantable system 100. For example, in some embodiments, an implantable system includes first, second, and third wearable devices and first, second, and third implantable substrates, and the first wearable device is configured to wirelessly transmit data only to and from a first implantable device. From the wearable devices 104, the data from all of the implantable substrates 102 present in the implantable system 100 can be configured to be transmitted either via wireless link, or a hardwired link to the EPU 108 where it can be processed and analyzed by one or more algorithms (e.g., a machine learning classifier or model). In some embodiments, the two or more implantable substrates are in contact with two or more different muscles. In some embodiments, the two or more implantable substrates are in contact with two or more different portions of the same muscle.

Before an algorithm can be used to process and classify incoming data, it must be trained. In some embodiments, training of the algorithm initially takes place on the EPU 108 (e.g., on a smartphone) or on the cloud. Once the algorithm has been trained, the trained algorithm is configured to process the input data in real-time, to control the peripheral device 110. The trained algorithm is configured to receive data and produce control outputs for the peripheral device 110, such as, but not limited to, continuous joint angles, discrete gestures, or other control parameters. In some embodiments, the peripheral device 110 is a prosthesis, a prosthetic device including an actuatable joint, an exoskeleton, an orthotic, and/or an exosuit. In some embodiments, the prosthesis is, but is not limited to, a robotic limb prosthesis (e.g., a robotic arm or leg prosthesis), a robotic hand prosthesis, and/or a robotic foot prosthesis. In some embodiments, the exoskeleton is, but is not limited to, a hip exoskeleton, a knee exoskeleton, an ankle exoskeleton, and/or a multiple joint exoskeleton. In some embodiments, the orthotic is, but is not limited to, a robotic foot orthotic, a robotic leg orthotic, a robotic ankle orthotic, a robotic knee brace, a robotic arm brace, a robotic leg brace. In some embodiments, the exosuit is, but is not limited to, a soft wearable robot composed of a textile. In some embodiments, the exosuit excludes an external rigid structure.

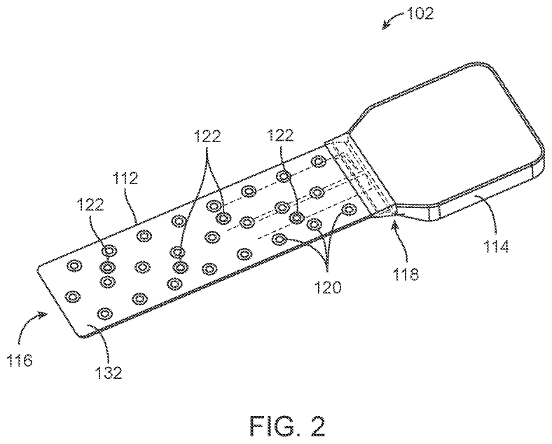

Referring to FIG. 2, the implantable device 102 is configured to be implanted in a subject, on the surface of one or more muscles of the subject. In some embodiments, the muscle is a skeletal muscle or a portion thereof. In some embodiments, the implantable device 102 is implanted in the subject such that the implantable device 102 is in direct contact with at least a portion of a fascia of the muscle, an epimysium of the muscle, a perimysium of the muscle, an endomysium of the muscle, a fascicle of the muscle, a muscle fiber, a tendon, a blood vessel of the muscle, a nerve of the muscle, or any combination thereof. In some embodiments, the fascia is a deep fascia of the muscle. In some embodiments, the deep fascia is an aponeurotic fascia and/or an epimysial fascia. In some embodiments, the implantable device 102 is implanted in the subject such that the implantable device 102 is in direct contact with at least a portion of loose connective tissue of the muscle. In some embodiments, the implantable device 102 is implanted in the subject such that the implantable device 102 is in direct contact with at least a portion of a surface of a fasciculus of the muscle.

In some embodiments, the implantable device 102 can be inserted under the skin through one or more small incisions (e.g., an incision having a length of about 0.5 centimeters (cm) to about 5 cm). For example, a small flexible camera can be placed at the tip of an insertion tool to provide the surgeon with a clear view of where the insertion tool is located in space to ensure accuracy and safety during pocket formation through a limited number of (e.g., one or more) incisions. Once the proper implant pocket length is achieved, the implantable device 102 can then be inserted into the implant pocket and deployed onto the surface of one or more muscles. In some embodiments, the implantable device 102 is not fixedly secured to the muscle. In some embodiments, at least a portion of the implantable device 102 can be secured in place via one or more sutures, surgical glues, or physical anchoring features of the implantable device 102 used to fix the implantable device 102 to the underlying or overlying tissues. In some embodiments, the implantable device 102 is configured to be sterilized (e.g., via autoclaving, gas sterilization, gamma radiation, etc.) prior to implantation.

The implantable device 102 includes an implantable substrate 112 and an electronic module 114 that are configured to operatively connect to each other, for example via a connector and/or a feedthrough architecture. The implantable substrate 112 is an elongated, generally flat substrate or strip having a proximal end 116 and a distal end 118. The implantable substrate 112 (e.g., an implantable sensor array substrate) includes one or more sensors (e.g., EMG sensors) 120, one or more reference electrodes 122, and an interconnect to electrically bond the one or more sensor pads 120 (e.g., EMG sensors or EMG electrodes) at the distal end 118. In some embodiments, the implantable substrate does not include one or more reference electrodes and/or biasing electrodes. In some embodiments, the reference electrodes 122 are biasing electrodes. In some embodiments, the implantable substrate 112 includes one or more reference electrodes and one or more biasing electrodes. In some embodiments, the sensors 120 are sensor pads. The electronic module 114 includes an opposing, second mating connector (e.g., a male or female connector) or feature configured to connect to the mating portion of the first connector of the implantable substrate 112. The electronic module 114 may further includes a case that houses the electronic components. In some embodiments, the electronic module may not further include a case that houses the electronic components. Instead, the electronic module may include a protective coating using technologies such as Atomic Layer Deposition (ALD) or Parylene C coating.

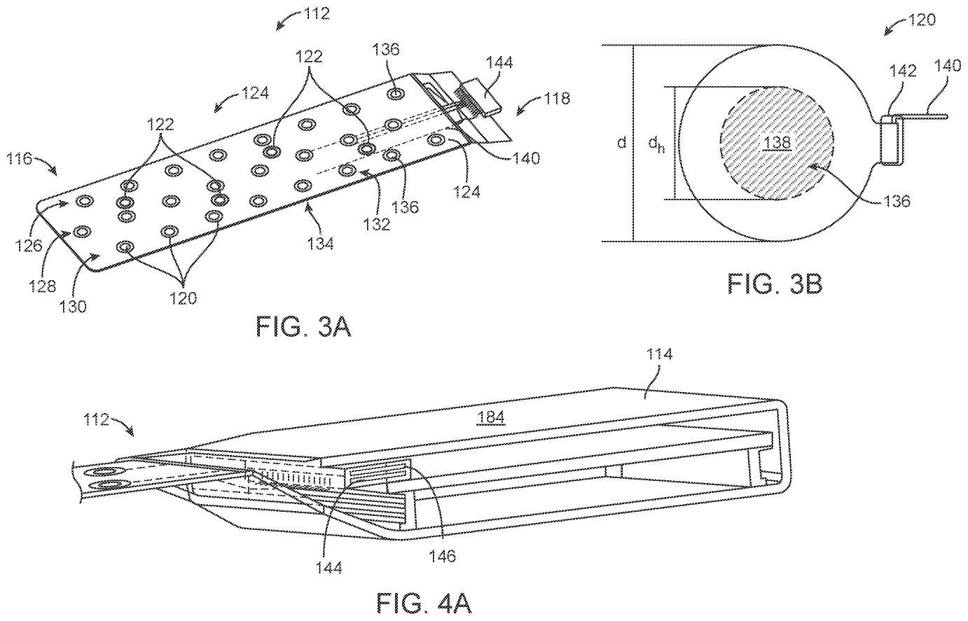

Referring to FIG. 3A, the implantable substrate 112 includes an electrode array 124 having three rows and eight columns of sensors 120 arranged in a grid configuration, for a total of twenty four sensors 120. The first row 126 and the third row 130 of sensors 120 are laterally aligned while the second row 128 of sensors 120 is longitudinally offset from the first and third rows 126, 130. The electrode array 124 further includes a pair of reference electrodes 122 that are staggered between the first and second rows 126, 128 and a pair of reference electrodes 122 that are staggered between the second and third rows 128, 130, for a total of four reference electrodes 122. The reference electrodes 122 are configured to be used as reference and bias drive. In some embodiments, the electrode connections can be reconfigured in situ. For example, in some embodiments, this can be implemented using analogue switches and/or multiplexers, which are controlled by a microcontroller. In some embodiments, the degree of reconfigurability depends on which exact components with suitable parameters can be sourced. For example, in some embodiments, fewer electrode configuration options can be implemented with single-pole, double-throw switches compared to a full switch matrix.

The center of each sensor 120 is about equidistant from the center of each of the neighboring sensors 120. In some embodiments, two or more sensors 120 are spaced equidistantly from a center of each of the two or more sensors 120. In some embodiments, the center-to-center sensor 120 spacing is about 10 mm. In some embodiments, the distance between the center of each sensor 120 and the center of an immediately adjacent sensor 120 is about 10 mm.

Alternative numbers of columns and rows may be employed. For example, in some embodiments, 4 or more electrodes are distributed into multiple rows and multiple columns. Also, every row need not contain the same number of columns. For example, an implantable substrate can include a design having one or more rows that include 10 columns of electrodes while additional rows can include 4 or more rows of electrodes to enable a greater amount of electrical field resolution.

The sensors 120 are biocompatible, electroconductive electrodes that are configured to contact a surface of a muscle in a subcutaneous, subadipose, or subfascial area of the subject and are configured to measure an electrical biopotential of the muscle. In some embodiments, the sensors 120 are EMG sensors. In some embodiments, the electrode array 124 includes about 4 to about 30 sensors 120. In some embodiments, the electrode array 124 includes about 35 to about 50 sensors 120. In some embodiments, the sensors 120 are platinum-iridium alloy electrodes. In some embodiments, the sensors 120 are carbon-based electrodes. In some embodiments, the sensors 120 are any other suitable type of biocompatible and/or bioinert metal such as titanium, or a biocompatible and/or bioinert polymer such as PEDOT (poly 3,4-ethylenedioxythiophene). In some embodiments, the reference electrodes 122 are platinum iridium electrodes. In some embodiments, the reference electrodes 122 are carbon-based electrodes. In some embodiments, the reference electrodes 122 are any other suitable type of biocompatible and/or bioinert metal such as titanium, or a biocompatible and/or bioinert polymer such as PEDOT (poly 3,4-ethylenedioxythiophene). In some embodiments, the sensors 120 are configured to have an impedance ranging from about 0.4 kiloOhm (kOhm) to about 1 MOhm (e.g., about 0.4 kOhm to about 0.5 kOhm, about 0.4 kOhm to about 0.6 kOhm, about 0.4 kOhm to about 0.7 kOhm, about 0.4 kOhm to about 0.8 kOhm, about 0.4 kOhm to about 0.9 kOhm, or about 0.7 kOhm to about 1 kOhm, about 1 kOhm to about 100 kOhm, about 100 kOhm to about 250 kOhm, about 100 kOhm to about 500 kOhm, about 100 kOhm to about 1 MOhm, about 500 kOhm to about 1 MOhm, about 1 kOhm to about 1 MOhm, or about 100 kOhm to about 500 kOhm) at 1 KHz.

The sensors 120 and reference electrodes 122 along with their wires 140 are embedded within the implantable substrate 112. The implantable substrate 112 is composed of a flexible and bioinert and/or biocompatible material. In some embodiments, the implantable substrate 112 is composed of silicone. Non-limiting examples of materials that the implantable substrate can be composed of include polymer-based materials (such as but not limited to silicone, liquid crystal polymer, or shape memory polymer) and a thin-film substrate coated with one or more biocompatible insulators (such as but not limited to silicone-carbide, silicone-oxide, or silicone-nitride). In some embodiments, the implantable substrate is configured to wrap around a muscle. In some embodiments, the implantable substrate is configured to wrap around a tissue having a generally cylindrical or tubular structure (e.g., a muscle of a limb). In some embodiments, the implantable substrate is configured to wrap around a circumference of one or more muscles of the subject when implanted.

The implantable substrate 112 has a top surface 132 and a bottom surface 134 opposing the top surface 132. The top surface 132 includes the sensors 120, and the bottom surface 134 includes the reference electrodes 122. In some embodiments, the sensors 120 are embedded within the top surface 132, and the reference electrodes 122 are embedded within the bottom surface 134. The top surface 132 is configured to be in contact with the muscle of the subject and defines one or more holes 136 to expose the sensors 120, thereby facilitating sensor 120-to-muscle contact. Referring to FIG. 3B, the sensors 120 have circular shape that is concentric with the holes 136. The sensors 120 have a diameter d of about 4 millimeters (mm), and the holes 136 have a diameter dₕ of about 2 mm. In some embodiments, the sensors have a diameter d that is larger than the diameter dₕ of the holes 136. In some embodiments, the diameter of the sensors 120 is about 50% to about 60% (e.g., about 50% to about 55% or about 55% to about 60%) larger than the diameter of the holes 136. The sensor 120 has an exposed area 138 that is configured to contact a muscle of the subject and is about 50% to about 60% larger than the area of the sensor 120. In some embodiments, the exposed area 138 can include a visual marker (e.g., a number or letter) that identifies one or more of the sensors 120 and the reference electrodes. In some embodiments, the top and bottom surfaces 132, 134 include a visual marker (e.g., a number or letter) or are colored differently to be distinguished from each other.

The wire 140 of each sensor 120 and reference electrode 122 is laser welded to the surface of its corresponding sensor 120 or reference electrode 122 at a laser weld joint 142. The sensor 120 or reference electrode 122 and the laser welded interface is encapsulated in a bioinert and/or a biocompatible material (e.g., silicone) to protect the electrical connection from the environment. The wire 140 can be composed of but is not limited to a conductive polymer, metal alloy, or carbon-based material.

Referring again to FIG. 3A, the implantable substrate 112 typically has a length (e.g., in a direction extending from the proximal end 116 of the implantable substrate 112 to the distal end 118 of the implantable substrate 112) of about 10 mm to about 300 mm and a width (e.g., extending across the lateral edges of the implantable substrate 112 of about 10 mm to about 200 mm. The implantable substrate 112 typically has a total thickness of about 0.5 mm to about 5 mm, providing the implantable device 102 with a film-like substrate having increased flexibility, which may be less noticeable to the subject when the implantable device 102 is implanted. The implantable substrate 112 has a generally rectangular shape with rounded edges; however, the implantable substrate can have any other suitable shape. In some embodiments, the implantable substrate 112 is sized to be wrapped around one or more muscles of a subject at a subcutaneous, subadipose, or subfascial depth.

The implantable device further includes a connector 144 at the distal end 118 of the implantable substrate 112. In some embodiments, the connector 144 is a male connector. In some embodiments, the connector 144 is a female connector. In some embodiments, the connector 144 is a pin connector. In some embodiments, the connector 144 is a pigtail or mating unit designed to feed into and join with a receiving unit via hermetically enclosed physical contact. In some embodiments, the connector 144 is bare wire 140. The wires 140 leading from each of the sensors 120 and reference electrodes 122 are affixed to the connector 144 via laser welding (or some alternative means of bonding), thereby fixedly securing the sensors 120 and the reference electrodes 122 to the connector 144. When the connector 144 is composed of bare wire 140, the wire 140 is laser welded or bonded in some fashion directly to the connector 146.

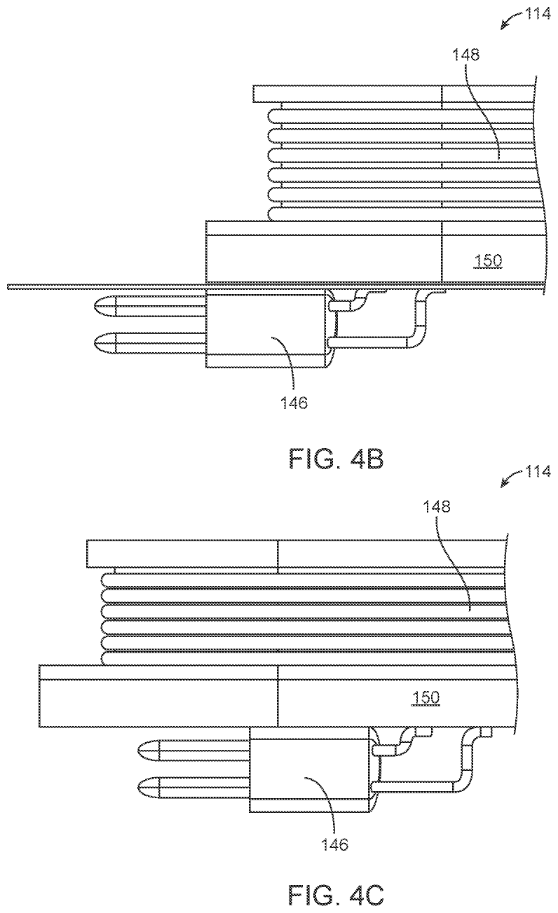

Referring to FIGS. 4A-4C, the implantable substrate 112 includes an electronic module 114 including a connector 146 configured to connect to connector 144, thereby connecting the electronic module 114 to the implantable substrate 112. In some embodiments, the connector 146 is a female connector. In some embodiments, the connector 146 is a male connector. In some embodiments, the connector 146 is a socket connector. In some embodiments, the connect 146 is a pin connector. In some embodiments, the connector 146 is a feedthrough connector.

The electronic module 114 further has a case 184 defining an enclosed space that houses one or more components (e.g., electronic components, a magnet, a sensor, and/or the like) of the implantable device. In some embodiments, the case 184 is composed of or coated with a biocompatible material and/or a bioinert material. In some embodiments, the case 184 is a hermetic enclosure that prevents fluid ingress and egress. In some embodiments, the case 184 is a rigid structure that provides physical protection for the components within it. In some embodiments, the case 184 is composed of a thermoplastic polymer (e.g., polyether ether ketone (PEEK)).

The components disposed within the open, interior space of the case 184 include a power receiver coil 148 configured to facilitate wireless inductive charging, wireless power transfer, and/or wireless communication of the implantable device, a printed circuit board (PCB) 150 containing electronic components configured to acquire, process, and/or transmit the sensor signals, a capacitor configured to store a minimal amount of charge or power to survive short power losses on the order of seconds, and a motion sensor configured to capture, measure, and/or transmit motion data of the implantable device. In some embodiments, the PCB 150 contains other electronic components such as, but not limited to, an optical sensor (e.g., a photoplethysmography (PPG) sensor, a peripheral oxygen saturation (SpO2) sensor, or the like), a pressure sensor, a force sensor, a humidity sensor, a temperature sensor, a chemical sensor, a location sensor, and/or a positioning sensor. In some embodiments, the motion sensor is an inertial measurement unit (IMU). In some embodiments, the motion sensor is a micro-electro-mechanical-system (MEMS)-based IMU. In some embodiments, the motion sensor is a combined accelerometer and gyroscope. The electronic module 114 does not include a battery or a Bluetooth® wireless communication component given that the wearable device provides these features.

Referring specifically to FIGS. 4B and 4C, in some examples, the connector 146 can be secured to a surface of the PCB 150 at various positions. For example, FIG. 4B illustrates the connector 146 being surface-mounted to the PCB 150 and being flush from the edge of the PCB 150 to the face of the connector 146. In another example, FIG. 4C illustrates the connector 146 being surface-mounted to the PCB 150 and being offset from the edge of the PCB 150. In some embodiments, the connector 146 is contained within the wall structure of the case 184. In some embodiments, the power receiver coil 148 and electronic components within the electronic module 114 are coated with parylene to waterproof these components and add dry lubricity. In some embodiments, the electronic module 114 includes two separate coils for wireless power (e.g., the power receiver coil 148) and wireless communications. The power receiver coil 148 is wound on a bobbin with the same outline as the PCB 150 and sits directly on it. In some embodiments, the power receiver coil 148 is embedded within the PCB 150 itself. In some embodiments, the power receiver coil 148 is embedded within the housing 152. The communications coil is a smaller solenoid-style coil mounted on a location inward on the PCB 150.

The implantable device uses a Near Field Magnetic Induction (NFMI) link to communicate with the wearable device. Sensor data (e.g., EMG data and/or motion data) is configured to be primarily sent from the implantable device to the wearable device over this link. Command signals and control signals are also configured to be transmitted over this link; for example, the supply voltage and current of the implantable device can be transmitted to the wearable device, and the wearable device can update settings for the wireless power transmitter over this link. Additionally, the wearable device is configured to transmit data to the implantable device over the NFMI link. In some embodiments, data is transferred directly over the power link (via a radiofrequency modulation scheme). In some embodiments, communication between the wearable device and the implantable device is accomplished via other suitable methods including, but not limited to, methods using galvanic, capacitive, ultrasound, optical, and molecular components.

In some embodiments, the implantable device is powered over a wireless power system using a magnetic link. In some embodiments, there is no significant energy storage on the implantable device; thus, the wireless link is configured to be on constantly while the system is in use. In some embodiments, the output voltage of the power receiver coil 148 is rectified and smoothed, resulting in an unregulated voltage from which all other power supplies are generated.

In some embodiments, the electronics module further includes an integrated current, voltage, and power measurement circuit configured to measure the voltage received by the implantable device and the current drawn by it. In some embodiments, measuring the voltage received and the current drawn enables an alignment assistance function of the wearable device and closed-loop power control, if necessary.

In some embodiments, the electronic module further includes a microcontroller (MCU) configured to capture data from an analogue front-end and forward it to the NFMI chip, along with system configuration and monitoring functions. In some embodiments, the MCU is a part of the NFMI chip. In some embodiments, the MCU is a component that is separate from the NFMI chip.

In some embodiments, the electronic module further includes an analog front end in order to perform analog signal processing such as filtering, noise reduction, and/or digitization of the signals.

In some embodiments, the electronics module further includes anti-aliasing circuits and/or buffers, multiplexers, and averaging circuits. In some embodiments, the electronics module may include additionally components for digital signal processing.

An implantable system may be substantially similar in construction and function in several aspects to the implantable systems 100 discussed above, but can include an alternative implantable device 103 instead of the implantable device 102. In some embodiments, the implantable device 103 may have different connectors and an electronic module having a rigid, hermetic case composed of ceramic. For example, the implantable device 103 may have a hermetic feedthrough connector. Such hermetic connectors and hermetic case can prevent ingress and egress of fluids when implanted in the body, can provide an electronic module with a slimmer profile, and can act as a protective casing for impact resistance. The implantable device 103 is respectively part of the implantable system 100 that otherwise includes a wearable device 104 that can be removably secured to a limb of the subject 106, an external processing unit (EPU) 108, and a peripheral device 110.

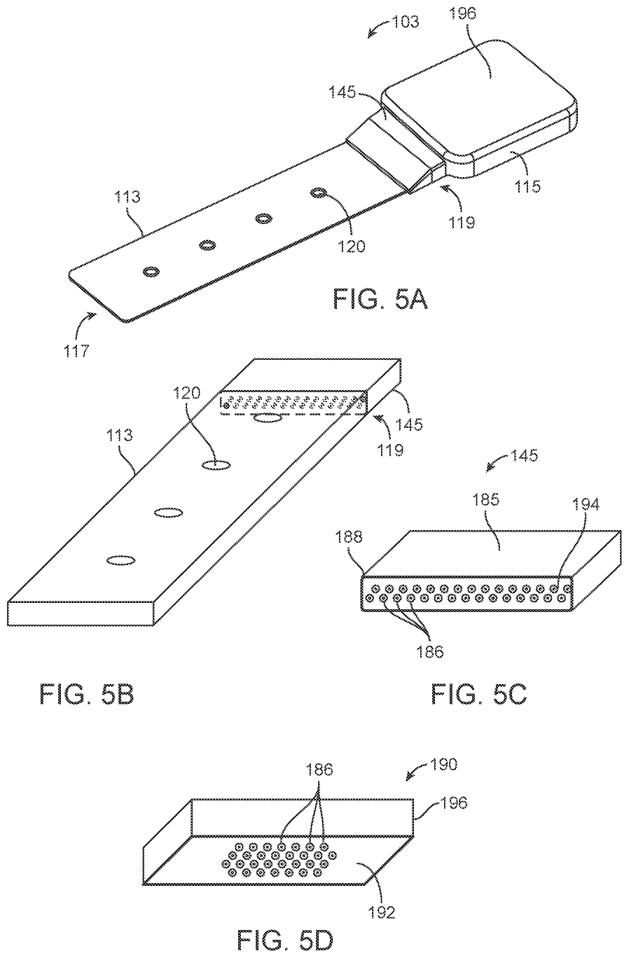

Referring to FIGS. 5A-5D, the implantable device 103 includes an implantable substrate 113 and an electronic module 115 that are configured to operatively connect to each other through a connector. The electronic module 115 includes a case 196 that houses the electronic components. The case 196 is composed of a ceramic material. The implantable substrate 113 is an elongated, generally flat substrate or strip having a proximal end 117 and a distal end 119. The implantable substrate 113 (e.g., an implantable sensor array substrate) includes one or more sensors (e.g., sensor pads or EMG sensors) 120, and one or more reference electrodes. The electronic module 115 includes a feedthrough connector 145 (e.g., a metal feedthrough connector) including a plurality of feedthroughs 186 defined by a side surface 194 and configured to directly connect to the wires of the sensors 120 and to the reference electrodes 122 of the implantable substrate 113 at the distal end 119. In some embodiments, the feedthrough connector 145 is a hermetic electrical contact feedthrough connector whereby the conductive leads of the array 112 converge into one or more pigtails that connect to the electronic module 115. In some embodiments, the feedthrough connector 145 is a metal feedthrough connector. The feedthrough connector 145 includes a case 185 with generally orthogonal dimensions including a metal flange 188 framing the side surface 194 for welding of a multi-part hermetic enclosure. The case 185 can be composed of a biocompatible material such as but not limited to ceramic, metal, thermoplastic, and/or any other rigid or semi-rigid polymer.

Referring specifically to FIG. 5D, an alternative feedthrough connector 190 includes a plurality of feedthroughs 186 defined by a bottom surface 192 instead of a side surface, as in the feedthrough connector 145 of FIGS. 5B and 5C.

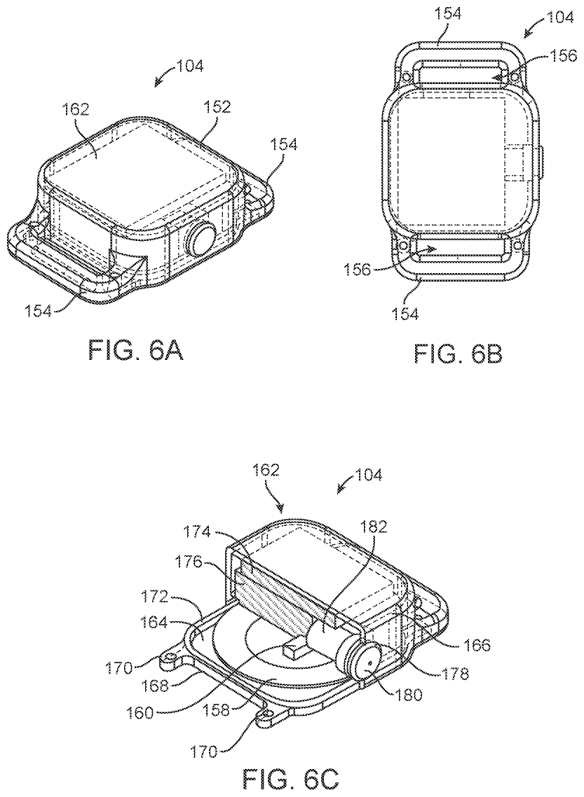

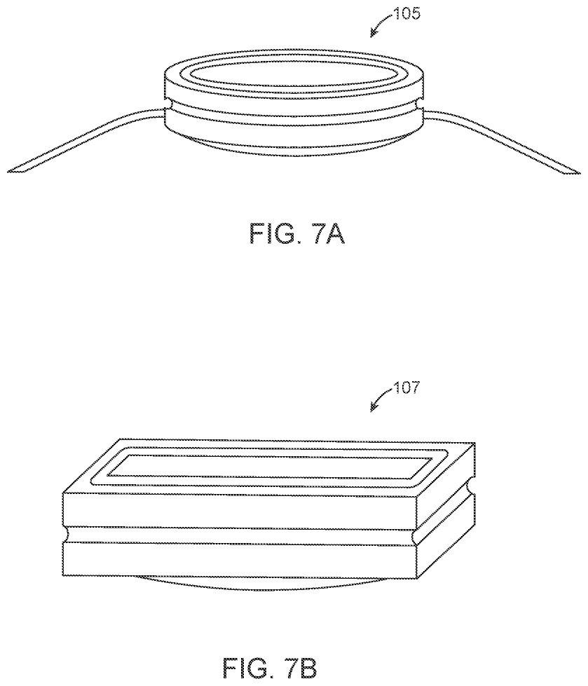

Referring to FIGS. 6A-6C, the wearable device 104 is an external module that is configured to communicate with and power the implantable device. The wearable device 104 is configured to send power to the implantable device and is configured to serve as a bridge between the implantable device and an external processing unit (e.g., a smartphone, a computer, a prosthesis, etc.).

The wearable device 104 has a generally square shape; however, the wearable device can have any suitable shape (e.g., a low profile disc or a low profile square), dimensions, and/or configuration. The wearable device 104 has a housing 152 defining an interior space configured to house one or more components (e.g., electronic components). The housing 152 includes a cover 166 that is configured to mate and be securely fixed to a base 168, thereby forming the enclosed space that houses the components. The cover 166 and base 168 are configured to be securely fixed to each other by a pair of retainers 170 (e.g., bolts). In some embodiments, other suitable methods of securely fixing the cover 166 to the base 168 (e.g., via a snap fit connection, an adhesive, a glue, etc.) can be used. To further secure the connection between the cover 166 and the base 168, the main body 162 includes a sealing member disposed around the four edges of the main body 162. The sealing member is configured to provide a water-resistant seal formed between the cover 166 and the base 168 when the cover 166 and the base 168 are coupled to form the interior space housing the components.

The housing 152 includes a pair of lugs 154. Each lug 154 is symmetrically arranged on opposing sides of the main body 162 of the housing 152. Each lug 154 is integrally connected to the main body 162 and extends outwardly from opposing edges of the main body 162. Each lug 154 defines a slot 156 configured to receive a strap that can be used to attach the wearable device 104 to a subject, for example. The components disposed within the interior space of the housing 152 include, for example, a power transmitter coil 158 configured to power the implantable device over the wireless link via an inductive magnetic field, a communication coil 160 configured to facilitate wireless communication, and a battery 174 that is self-contained and configured to supply power to the electronic components of the wearable device 104. In some embodiments, the housing 152 does not contain lugs 154 and slots 156, but rather contains structural features designed to snap on, slide in, or affix a strap that can be used to attach the wearable device 104 to a subject.

The power transmitter coil 158 is configured to sit on an internal surface 164 of the base 168, within the internal space defined by the main body 162 of the housing 152. As described above, the wearable device 104 is configured to power the implantable device over a wireless link. The power transmitter coil 158 includes an amplifier to drive the coil that will generate a magnetic field. In some embodiments, the magnetic link of the power transmitter coil 158 is configured to use an operating frequency that is greater than an operating frequency to be used by the NFMI communications link to increase separation and prevent undesirable electromagnetic interference. In some embodiments, the amplifier is a high efficiency amplifier. In some embodiments, the amplifier is configured to keep the end-to-end efficiency of the wireless power link as high as possible, thereby extending the battery life as much as possible. In some embodiments, the power transmitter coil 158 is configured to be controllable to implement a closed loop control of the wireless power link, if required.

At least a portion of the communication coil 160 is disposed on an internal surface 164 of the base 168, within the internal space defined by the main body 162 of the housing 152, and in close proximity to the power transmitter coil 158, as shown in FIG. 6C. The configuration and construction of the communication coil 160 is similar to the communication coil in the implantable device.

The battery 174 is disposed over the PCB 176 within the internal space defined by the main body 162 of the housing 152. The battery 174 is a rechargeable battery configured to be charged when an external power source is connected to it. In some embodiments, the battery 174 is a lithium-ion battery. In some embodiments, the battery 174 is a pouch cell battery with built-in protection circuitry. In some embodiments, the battery 174 is a prismatic cell with built-in protection circuitry. In some embodiments, the battery 174 is a lithium-ion pouch cell or prismatic cell battery with built-in protection circuitry. In some embodiments, the battery 174 has a battery capacity configured to support a 2-hour data acquisition time and an additional hour for preparation and alignment. In some embodiments, the battery 174 is configured to support about 2 hours (h) to about 24 hours (e.g., about 2 h to about 3 h, about 2 h to about 4 h, about 2 h to about 5 h, about 2 h to about 6 h, about 2 h to about 7 h, about 2 h to about 8 h, about 2 h to about 9 h, about 2 h to about 10 h, about 2 h to about 11 h, about 2 h to about 12 h, about 2 h to about 14 h, about 2 h to about 16 h, about 2 h to about 18 h, about 2 h to about 20 h, about 2 h to about 24 h, about 12 h to about 24 h) of data acquisition time. In some embodiments, the battery 174 is sized to fit within the enclosed space defined by the main body 162. In some embodiments, the battery 174 is a cylindrical cell having a reduced surface area with respect to a pouch cell or a prismatic cell battery. In some embodiments, the battery 174 is a flexible and conformal substrate to accommodate unconventional form factors.

In some embodiments, the wearable device 104 can be fully operational while simultaneously charging the battery 174 when connected to an external power supply. In some embodiments, this configuration is not foreseen to be necessary in a normal usage scenario, however, this configuration is configured to enable the run time of the wearable to be easily extended (e.g., by connecting it to an external power bank).

As described above, the wearable device 104 uses a Near Field Magnetic Induction (NFMI) link to communicate with the implantable device. Sensor data (e.g., EMG data and/or motion data) from the implantable device is configured to be received by the wearable device 104 over this link. Command signals and control signals are also configured to be transmitted over this link; for example, the supply voltage and current measurements of the implantable device for alignment and closed loop power control, if required. In some embodiments, the wearable device 104 can update settings for the wireless power transmitter over this communication link.

In some embodiments, the wearable device 104 communicates with an EPU (e.g., a personal computer (PC), a smartphone, or the like) via a short range communications link (e.g., a Bluetooth® link). In some embodiments, the wearable device 104 includes an integrated Bluetooth® module or a Bluetooth® chipset to enable such communication. In some embodiments, the wearable device 104 is configured to transmit sensor data (e.g., motion data and/or EMG data) to the EPU over the Bluetooth® link. In some embodiments, the PCB 176 includes a microcontroller configured to receive data (e.g., sensor data) sent from the implantable device over the NFMI link and is configured to forward the data to a EPU (e.g., PC, smartphone, or the like) via the Bluetooth® link. In some embodiments, the microcontroller is configured forward system configurations and monitoring functions to the EPU via the Bluetooth® link. In some embodiments, the PCB 176 includes a decoder configured to decode the EMG signals on the wearable.

As shown in FIGS. 6A-6C, the wearable device 104 includes a connector 178 configured to allow access to an external device (e.g., an external power supply and/or an EPU). Thus, the connector 178 is configured to enable charging of the battery 174 as well as configuration and debugging of the assembled wearable device 104 in the field. The connector 178 includes a connector cap 180 and a shaft 182. The connector cap 180 is external to the housing 152 and is removably coupled to the shaft 182. The shaft 182 extends through the housing 152, within the enclosed area defined by the main body 162. As the connector 178 is a major potential ingress point in an otherwise sealed device in a harsh environment, the connector 178 provides an appropriate level of ingress protection and robustness. In some embodiments, the connector cap 180 can be a blanking cap, a plug, or a push-pull connector. In some embodiments, the connection between the connector cap 180 and the shaft 182 is a watertight connection and/or a vacuum tight connection. In some embodiments, the connector 178 is configured to support signal transmission (e.g., ground signals, power transmission, debugging signals, or the like) via a USB having a modified terminal configured to couple with the connector 178. In some embodiments, the connector 178 includes about 4 pins to about 12 pins.

The wearable device 104 may have a variety of ways of providing feedback to the user about particular conditions (e.g., if there is an active alignment assistance or a need to communicate a state such as, but not limited to, Bluetooth® pairing, confirmation of power on and/or off. In some embodiments, the feedback is a direct visual feedback, where the wearable device 104 incorporates an indicator light (e.g., a light emitting diode (LED) along with a light pipe/guide) disposed on the outside of the housing 152. In some embodiments, the indicator light is disposed within the internal space defined by the main body 162 of the housing 152. In some embodiments, the wearable device 104 provides tactile feedback, where the wearable device 104 can vibrate, buzz, or otherwise stimulate the user's sense of touch. In some embodiments, the wearable device 104 provides auditory feedback, where the wearable device 104 can beep, click, or otherwise generate any other suitable type of sound. In some embodiments, the wearable device 104 simultaneously provides visual, tactile, and auditory feedback.

In some embodiments, the wearable device 104 includes a Hall effect switch configured to turn on in the presence of a magnet or magnetic field and turn off when the magnet or magnetic field is removed. For example, if it is necessary for the user to interact directly with the wearable device 104 (e.g., to wake it up from a low-power mode, initiate Bluetooth® pairing, or the like), a Hall effect switch can be configured to detect a magnet that is brought close to a defined location near the enclosure, defined by the main body 162, where the Hall effect switch is located. In some embodiments, using a Hall effect switch instead of a physical switch advantageously keeps the user interaction a contactless one, where the enclosure can remain completely sealed, without risking the creation of an ingress path via a switch.

In some embodiments, a capacitive sensor can also be used to switch between modes in place of the Hall effect switch. In some embodiments, the capacitive sensor is configured to measure the change in capacitance when the user's finger is brought near the capacitive sensor. Like the Hall effect sensor, this capacitive sensor makes the user interaction contactless, where the enclosure can remain completely sealed, thereby improving fluid ingress protection.

An implantable system may be substantially similar in construction and function in several aspects to the implantable systems 100 discussed above, but can include a first wearable device 105 or a second wearable device 107 instead of the wearable device 104. In some embodiments, the first or second wearable devices 105, 107 may have a different shape and/or reduced dimensions with respect to the wearable device 104 shown in FIGS. 6A-C. For example, the first wearable device 105 has a circular shape and the second alternative wearable device 107 has a rectangular shape. Such exemplary shapes and reduced dimensions can provide a wearable device with a slimmer profile, which can enhance the formfactor and comfort of the wearable device 104. The first wearable device 105 or a second wearable device 107 is respectively part of the implantable system 100 that otherwise includes an implantable substrate 102 (or an implantable substrate 103), an external processing unit (EPU) 108, and a peripheral device 110.

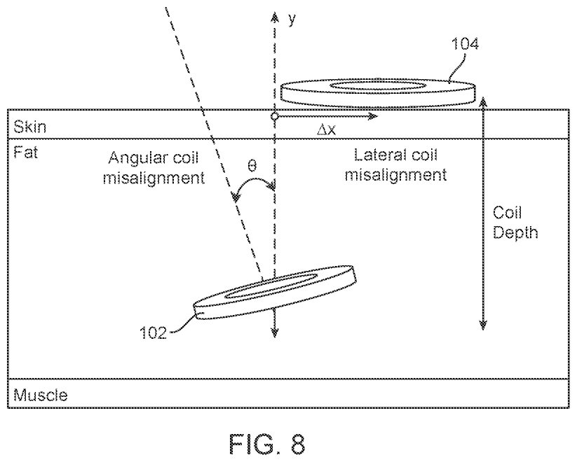

FIG. 8 illustrates examples of angular coil misalignment, lateral coil misalignment, and depth between the wearable device 104 on the skin surface and an implanted implantable device 102 in close proximity to a muscle surface. The implantable device 102-to-wearable device 104 interface is between the electronic components and magnet of the implantable device 102 to the external bridging hardware of the wearable device on the skin surface. This interface is important to ensure reliable data collection and transmission between the implantable device 102 and the wearable device 104. The interface also includes a power link between the power receiver coil and the power transmitter coil that is critical to power up the implantable device 102.

In some embodiments, the lateral coil misalignment can be defined as the distance x between the center of the power receiver coil of the implantable device 102 and the rim or an edge of the power transmitter coil of the wearable device 104. In some embodiments, the implantable device 102 and wearable device 104 can have a lateral coil misalignment of about 5 mm to about 15 mm at most (e.g., about 5 mm to about 6 mm, about 5 mm to about 7 mm, about 5 mm to about 8 mm, about 5 mm to about 9 mm, about 5 mm to about 10 mm, about 5 mm to about 11 mm, about 5 mm to about 12 mm, about 5 mm to about 13 mm, about 5 mm to about 14 mm, about 5 mm to about 10 mm, or about 10 mm to about 15 mm) for ideal functioning of the implantable device 102 and the wearable device 104 (e.g., having reliable data transmission and collection and powering up of the implantable device 102).

In some embodiments, the angular coil misalignment can be defined as the angle theta (θ) of the implantable device 102 relative to the Y-axis, which extends through the center of the power receiver coil of the implantable device 102 and is adjacent to the rim or an edge of the power transmitter coil of the wearable device 104. In some embodiments, the implantable device 102 and wearable device 104 can have an angular coil misalignment of about 5 degrees to about 15 degrees at most (e.g., about 5 degrees to about 6 degrees, about 5 degrees to about 7 degrees, about 5 degrees to about 8 degrees, about 5 degrees to about 9 degrees, about 5 degrees to about 10 degrees, about 5 degrees to about 11 degrees, about 5 degrees to about 12 degrees, about 5 degrees to about 13 degrees, about 5 degrees to about 14 degrees, about 5 degrees to about 15 degrees, about 5 degrees to about 10 degrees, or about 10 degrees to about 15 degrees) in any direction for ideal functioning of the implantable device 102 and the wearable device 104 (e.g., having reliable data transmission and collection and powering up of the implantable device 102).

In some embodiments, the coil depth can be defined as the subcutaneous depth of the implantable device 102, once implanted, relative to the skin surface and to the wearable device 104. In some embodiments, the implantable device 102 can have a coil depth ranging of about 10 mm to about 50 mm at most (e.g., about 10 mm to about 11 mm, about 10 mm to about 12 mm, about 10 mm to about 13 mm, about 10 mm to about 14 mm, about 10 mm to about 15 mm, about 10 mm to about 16 mm, about 10 mm to about 17 mm, about 10 mm to about 18 mm, about 10 mm to about 19 mm, about 10 mm to about 20 mm, about 10 mm to about 21 mm, about 10 mm to about 22 mm, about 10 mm to about 23 mm, about 10 mm to about 24 mm, about 10 mm to about 25 mm, about 10 mm to about 26 mm, about 10 mm to about 27 mm, about 10 mm to about 28 mm, about 10 mm to about 29 mm, about 20 mm to about 30 mm, about 15 mm to about 30 mm, about 20 mm to about 40 mm, about 20 mm to about 50 mm, about 30 mm to about 40 mm, about 30 mm to about 50 mm, or about 40 mm to about 50 mm) for ideal functioning of the implantable device 102 and the wearable device 104 (e.g., having reliable data transmission and collection and powering up of the implantable device 102).

An implantable system 200 may be substantially similar in construction and function in several aspects to the implantable system 100 discussed above, but can include an intermediate physical wireless access point that is configured to act as a processor for data decoding and storage and as a relay station for all of the wireless and/or wired component communication and synchronization. In some embodiments, the intermediate physical wireless access point is a communication controller 211.

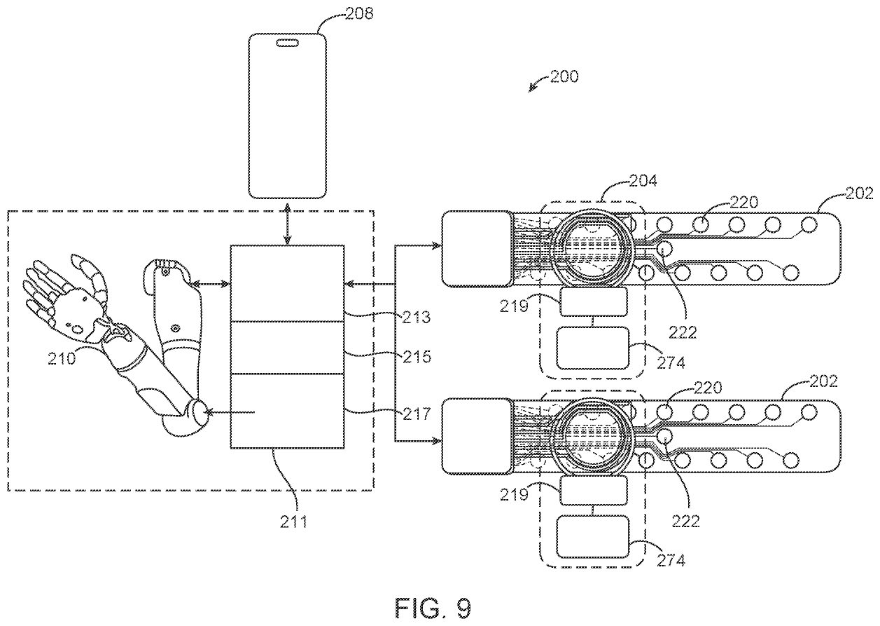

FIG. 9 illustrates an example of an implantable system 200 that can be used to control an orthopedic device (e.g., a prosthesis and/or an exoskeleton) via the detection of EMG signals generated by one or more muscles of a subject. The implantable system 200 includes one or more implantable devices 202 that can be implanted in a pocket formed within the subcutaneous or subadipose or subfascial anatomical planes in a subject, a wearable device 204 that can be removably secured to a limb of the subject, an external processing unit (EPU) 208 (e.g., a mobile device or a mobile phone), a peripheral device 210 (e.g., a prosthetic device and/or an exoskeleton), and the communication controller 211.

The communication controller 211 includes a communication module 213, a processor 215, and an energy storage unit 217. In some embodiments, the communication module 213 is a universal asynchronous receiver/transmitter-controlled area network (UART-CAN) module. The communication module 213 is a converter module that facilitates wireless and/or wired communications between one or more of the implantable device 202, the peripheral device 210, and the EPU 208. The co-processor 215 is configured to decode and/or store data generated by one or more of the implantable device 202, the peripheral device 210, and the EPU 208. The energy storage unit 217 is configured to store power received from a power source. The energy storage unit 217 is configured to provide power to one or more of the communication controller 211, the implantable device 202, and the peripheral device 210. In some embodiments, the implantable system 200 includes two or more energy storage units 217. In some embodiments, each energy storage unit 217 is configured to provide power to separate components individually. In some embodiments, the implantable system 200 includes a single energy storage unit pack. In some embodiments, the energy storage unit 217 is a battery. In some embodiments, the communication controller 211 can operate with the inclusion or the exclusion of the EPU 208.

The implantable device 202 includes one or more EMG sensors that can detect EMG signals generated by one or more muscles of the subject. The wearable device 204 can wirelessly connect to, power, and recharge the implantable device 202 when placed on a skin surface of the subject, in the vicinity of the implantable device 202. In this manner, the wearable device 204 can wirelessly transmit data (e.g., EMG data, motion data, a configuration parameter, a status parameter, and/or other types of sensor data) to and from the implantable device 202 via a wireless induction link, or other type of wireless communication system. In some embodiments, the wireless data transmission takes place via any suitable wireless connection such as, but not limited, to Wi-Fi, Bluetooth®, near field communication (NFC), near field magnetic induction (NFMI), or any combination thereof. In some embodiments, the wireless power transmission can take place via NFC.

In some embodiments, the data is configured to be transmitted from the wearable device 204 to the EPU 208 or the peripheral device 210 using the communication controller 211. The EPU 208 can be a smartphone, mobile device, or other portable processing unit. In some embodiments, the EPU 208 can be a part of the wearable device 204 or the peripheral device 210. On the EPU 208, the data is received from the communication controller 211 where it may further be processed and/or analyzed by one or more algorithms (e.g., a machine learning classifier or model, time-series signal processing).

As shown in FIG. 9, the implantable system 200 can include two or more implantable substrates 202 that can work as a cohesive system where the two or more implantable substrate 202 can wirelessly connect to, be powered by, and be recharged by two or more wearable devices 204. Each wearable device 204 includes a power module 219. The power module 219 is configured to enable the wearable device 204 to wirelessly charge the implantable device via NFC. In some embodiments, the power module 219 is configured to wirelessly charge (e.g., receive power) the wearable device 204 via NFC. In some embodiments, the power modules 219 of the wearable devices 204 can either be separate independent modules or incorporated components into the physical structure of the peripheral device 210 (e.g., of a prosthesis socket). In some embodiments, the power modules 219 of two or more wearable devices 204 are wired together. In some embodiments, the power modules 219 of two or more wearable devices 204 are not wired together and are wireless. In some embodiments, the power modules 219 of two or more wearable devices 204 are configured to wirelessly communicate with each other (e.g., to coordinate power optimization and/or minimize magnetic interference). In some embodiments, the two or more wearable devices 204 communicate with each other through a wired connection to the communication controller 211 and/or through a wireless link to the communication controller 211. In some configurations, the wearable devices 204 communicate directly between each other. The power module 219 includes an energy storage unit 274 that is configured to store energy received from a power source and configured to provide energy to the wearable device 204. In some embodiments, the energy storage unit 274 is a battery.

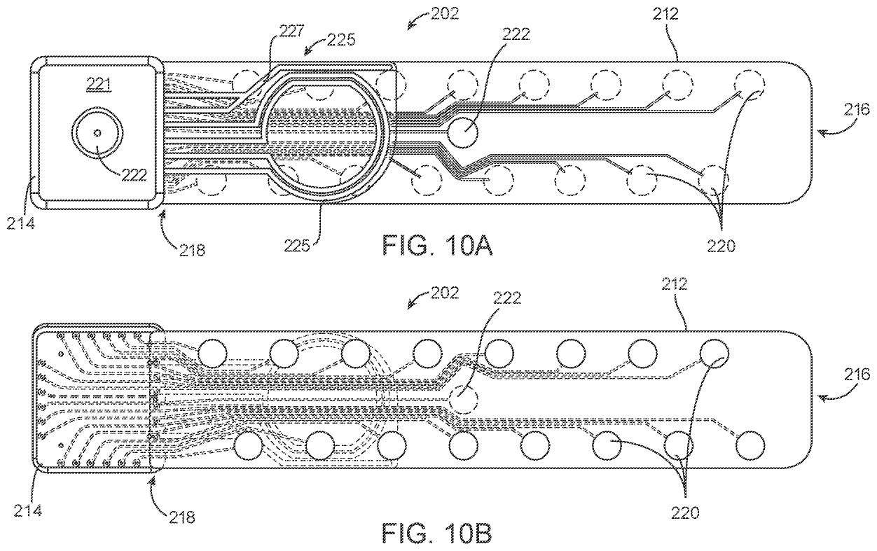

FIGS. 10A and 10B illustrate a top view and bottom view, respectively, of the implantable device 202. The implantable device 202 includes an implantable substrate 212 and an electronic module 214 that are configured to operatively connect to each other, for example via a connector and/or a feedthrough architecture. The implantable substrate 212 is an elongated, generally flat substrate or strip having a proximal end 216 and a distal end 218. The implantable substrate 212 (e.g., an implantable sensor array substrate) includes one or more sensors (e.g., EMG sensors or EMG electrodes) 220, one or more reference electrodes 222, and an interconnect to electrically bond the one or more sensor pads 220 (e.g., EMG sensors or EMG electrodes) at the distal end 218. In some embodiments, the reference electrodes 222 are biasing electrodes. In some embodiments, the implantable device 202 is subcutaneously implanted in a subject such that the surface of the reference electrode 222 that is in contact with the subcutaneous tissue faces towards a skin surface (e.g., is outward-facing), and the surface of the sensors 220 that is in contact with the subcutaneous tissue faces towards a muscle surface (e.g., is inward-facing). In some embodiments, the implantable substrate 212 includes one or more reference electrodes and one or more biasing electrodes. In some embodiments, the sensors 220 are sensor pads. In some embodiments, the implantable device 202 includes sixteen sensors 220 and one reference electrode 222.

The implantable substrate 212 includes an antenna 225 operatively connected to the electronic components of the electronic module 214. In some embodiments, the antenna 225 is a Wi-Fi antenna. The antenna 225 is configured to enable wireless communication and wireless power transfer between the implantable device 202 and other devices (e.g., the wearable device 204). The antenna 225 includes an antenna monopole 227 and an antenna loop 229. The antenna monopole 227 is configured to enable wireless communication between the implantable device 202 and other devices (e.g., the wearable device 204). The antenna loop 229 is configured to enable wireless power transfer (WPT) between the implantable device 202 and other devices (e.g., the wearable device 204). The electronic module 214 includes a capsule 221 that houses the electronic components. In some embodiments, the capsule 221 is composed of an inert, biocompatible material that is safe for human use (e.g., titanium). The capsule 221 includes an additional reference electrode 222. Either reference electrode 222 (e.g., the reference electrode 222 on the implantable substrate 212 or the reference electrode 222 in the capsule 221) can be used at a time or both reference electrodes 222 can be used simultaneously, to act as a reference to the sensors 220 on the implantable substrate 212.

### Methods of Using the Implantable System

The implantable system of the disclosure can be used to control a peripheral device (e.g., a prosthesis or exoskeleton) and can be used to translate, interpret, or convert gestures or sign language into speech or words. For example, in some embodiments, the methods of the disclosure include detecting sensor data (e.g., EMG sensor data and/or motion sensor data) from a subject using any of implantable devices disclosed herein. The method further includes wirelessly transmitting the sensor data from the implantable device to any of the wearable devices disclosed herein, wirelessly transmitting the sensor data from the wearable device to an external processing unit, processing the sensor data using one or more machine learning classifiers, and based on the processing step performed by the machine learning classifiers, generating one or more control outputs that lead to the translation, interpretation, conversion, and/or display of one or more gestures or sign language into an audible sound, speech, one or more words configured to be displayed on a screen (e.g., a screen of the EPU, a mobile device screen, a computing device screen, or the like), and/or one or more images configured to be displayed on the screen. In some embodiments, the audible sound, speech, words, and/or images are reproduced, broadcast, and/or displayed in a same device. In some embodiments, the audible sound, speech, words, and/or images are reproduced, broadcast, and/or displayed in one or more different and individual devices. In some embodiments, the audible sound, speech, words, and/or images are simultaneously reproduced, broadcast, and/or displayed in a device. In some embodiments, the device is operatively connected to the implantable system described herein.

Disclosed herein, in certain embodiments, are methods of controlling a peripheral device (e.g., a prosthesis, an exoskeleton, and/or an exosuit). The methods include detecting sensor data (e.g., EMG sensor data and/or motion sensor data) from a subject using any of implantable devices disclosed herein. The method further includes wirelessly transmitting the sensor data from the implantable device to any of the wearable devices disclosed herein, wirelessly transmitting the sensor data from the wearable device to an external processing unit, processing the sensor data using one or more machine learning classifiers, and based on the processing step performed by the machine learning classifiers, generating a control output for the peripheral device (e.g., a prosthesis, an exoskeleton, and/or an exosuit).

As disclosed above, the data received by the EPU 108 from the wearable device 104 is configured to be processed by one or more algorithms. In some embodiments, the algorithm is a machine learning classifier or machine learning model. In some embodiments, the algorithm is trained with and is configured to classify either raw sensor data or sensor data with a pre-processing feature extraction. This sensor data includes but is not limited to EMG data and motion sensor data (e.g., IMU data).

In some embodiments, the algorithm is a discrete classifier. In some embodiments, the discrete classifier includes a determined number of predetermined output classes, each of which represents a different state for a peripheral device (e.g., a prosthesis, an exoskeleton, and/or an exosuit), such as a gesture, a joint angle, or a movement for a prosthesis. In some embodiments, each of these output classes are mutually exclusive in their activation state, meaning that only one of the classes can be active at any time. In some embodiments, the discrete classifier can be paired with a proportional control system, where the discrete classifier determines which degrees of freedom are moving, and a proportional signal (e.g., the integral of the absolute value of the EMG signal) determines the speed or torque of the degrees of freedom in motion. In some embodiments, this discrete classifier can be an algorithm with a high number of hyperparameters, such as deep learning, or a low number of hyperparameters, like a logistic regression, linear discriminant analysis, or support vector machine classifier. In some embodiments, other suitable types of algorithms that can be used to create this type of model.

In some embodiments, the algorithm is a continuous classifier. In some embodiments, the continuous classifier includes a determined number of outputs that can be simultaneously active. In some embodiments, in the case of a control system for a prosthesis or exoskeleton, each output of the classifier controls a continuous value, such as, but not limited to, a joint angle, a torque, or an angular velocity of a single degree of freedom (DoF). In some embodiments, other suitable types of algorithms that can be used to create this type of model.

In some embodiments, the algorithm is trained before it processes and classifies sensor data. In some embodiments, the training of the algorithm takes place on an EPU (e.g., on a smartphone, tablet, computing device, or the like) or on the cloud and required data input from the user. Once the implantable device has been subcutaneously implanted in the user, and the user is wearing the wearable device, the user can begin the training process. In some embodiments, the training process starts by having the user connect her/his wearable device(s) to their EPU (e.g., a smartphone). In some embodiments, the EPU includes an executable program (e.g., a mobile application) that is configured to facilitate the training process. Once the wearable device is connected to the EPU, the user can open a training menu on the executable program (e.g., a mobile application) to begin the training. A display (e.g., a screen) of the EPU is configured to display a virtual representation of the peripheral device (e.g., a prosthesis and/or an exoskeleton) of the user.

To train the algorithms based on the sensor data of the user, the virtual representation of the peripheral device (e.g., a prosthesis and/or an exoskeleton) on the display shows a series of movements that the user must perform with her/his body (e.g., the movement is performed with a residual limb when the peripheral device is a limb prosthesis) to the best of their ability. In some embodiments, the implantable device is configured to capture sensor data (e.g., EMG signals and/or IMU signals) of the user and wirelessly transmit the sensor data to the wearable device. In some embodiments, the wearable device is configured to wirelessly transmit the sensor data to the EPU or the cloud. When the user has provided all of the required data, the system trains the algorithms and generates the model parameters, which are loaded into the model stored locally on the EPU. These parameters determine how the input information is transformed into the desired outputs. The user can then use the entire system (e.g., implantable device, wearable device, and EPU, including the trained algorithm) to control a virtual peripheral device on an EPU interface to practice using their system. In some embodiments, alternatively, the user can connect to a physical device (e.g., the prosthetic, the exoskeleton, an exosuit, or other peripheral device) and begin using their prosthetic, exoskeleton, or other peripheral device. For example, the EPU is then configured to transmit a control output to the prosthesis. In some embodiments, the control outputs include, but are not limited to, a joint angle, a torque, a discrete gesture, an angular velocity of a single degree of freedom (DoF), one or more words, and one or more images. In some embodiments, the output of a continuous model, for the control of a robotic device, may be one or more joint angles, voltage values, electric current values, and/or angular velocities. In some embodiments, this continuous output, for some other peripheral device (e.g., a smartphone) may be a volume level, brightness level, and/or any other adjustable range of continuous values (e.g., settings on a smartphone). In some embodiments, the output for a discrete model may be an integer, which corresponds to a gesture, word, phoneme, and/or image.

### Alternative Embodiments

While the above-discussed implantable devices and systems have been described and illustrated with respect to certain dimensions, shapes, arrangements, configurations, material formulations, and methods, in some embodiments, an implantable device, that is otherwise substantially similar in construction and function to the implantable devices previously described herein, may include one or more dimensions, shapes, arrangements, configurations, and/or materials formulations that are different from the ones discussed above or may be used with respect to methods that are modified as compared to the methods described above.

For example, while the implantable device 102 has been described and illustrated as excluding a battery or a Bluetooth® wireless communication component given that the wearable device provides these features, in some embodiments, an implantable device that is otherwise substantially similar in construction and function to the implantable device 102 may alternatively include an energy storage unit (e.g., a battery) such that the wearable device intermittently re-charges the implantable device. and/or a short-range communication link (e.g., a Bluetooth® wireless component), or some alternative component configured to enable wireless communications, to communicate directly with the external processing unit 108. For example, in some embodiments, this configuration may also include a wearable device that is otherwise substantially similar in construction and function to the wearable device 104, that alternatively may exclude a an energy storage unit (e.g., a battery) and/or a short range communication link (e.g., a Bluetooth® wireless component), or some alternative component configured to enable wireless communications, given that one or more of these components may be provided in the alternative implantable device. In some embodiments, in another example, the implantable system may not require a wearable device and may be a wearable-free, implantable system where the communications and power hardware are contained within and/or on the implantable device.

While the implantable systems and methods have been described and illustrated as including an EPU that is configured to wirelessly receive sensor data from the wearable device and is configured to wirelessly transmit a signal (e.g., a control output) to the peripheral device (e.g., a prosthesis, an exoskeleton, and/or an exosuit), in some embodiments, an implantable system that is otherwise substantially similar in construction and function to the implantable systems previously described may exclude an EPU. For example, in some embodiments, the wearable device is configured to wirelessly transmit the sensor data directly to the peripheral device, and the peripheral device is configured to process the sensor data and generate a control output in situ.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An implantable device, comprising:
an implantable substrate comprising a sensor configured to wirelessly detect and wirelessly transmit an electromyography (EMG) signal generated by one or more muscles of a subject, the implantable substrate being implanted in the subject and comprising a top surface and a bottom surface, both the top surface and the bottom surface being in contact with the one or more muscles of the subject, the sensor being embedded within the top surface and the bottom surface comprising one or more reference electrodes; and
a processor operatively coupled with the implantable substrate and configured to wirelessly receive and wirelessly transmit the EMG signal to an external processing unit.

2. The implantable device of claim 1, wherein the sensor comprises an EMG sensor and a motion sensor, wherein the EMG sensor and the motion sensor are configured to be inductively powered by an external power source.

3. (canceled)

4. (canceled)

5. The implantable device of claim 1, further comprising a second EMG sensor configured to detect and transmit the EMG signal generated by a second muscle of the one or more muscles of the subject, wherein the sensor is a first sensor configured to detect and transmit the EMG signal generated by a first muscle of the one or more muscles of the subject, wherein each of the first muscle and the second muscle is a skeletal muscle or a portion thereof.

6. (canceled)

7. The implantable device of claim 1, wherein the implantable device is coated with or encapsulated within a biocompatible material and/or a bioinert material, wherein the biocompatible and/or bioinert materials comprises at least one of a silicone material, a ceramic material, an epoxy material, a fabric, a cellular scaffold, an acellular scaffold, a plastic material, a metallic material, a carbon derivative, a synthetic biological tissue, a polymer, a meshed material.

8. (canceled)

9. (canceled)

10. The implantable device of claim 1, wherein the processor is configured to wirelessly receive the EMG signal from the sensor and is configured to wirelessly transmit the EMG signal to a wearable device.

11. (canceled)

12. The implantable device of claim 10, wherein the wearable device comprises a self-contained battery; and a power transmitter configured to wirelessly transmit energy to the implantable device via an inductive magnetic field, wherein the power transmitter further comprises an amplifier configured to amplify a magnetic signal, and wherein the self-contained battery is rechargeable, wherein the wearable device further comprises a power receiver.

13. (canceled)

14. (canceled)

15. (canceled)

16. The implantable device of claim 10, wherein the processor is a first processor, and wherein the wearable device comprises a second processor configured to: i) wirelessly receive the EMG signal from the first processor, and ii) wirelessly transmit the EMG signal to a computing device, wherein the second processor receives the EMG signal from the first processor via a near field magnetic inductive link, and wherein the second processor transmits the EMG signal to the computing device via a short-range communications link.

17. (canceled)

18. (canceled)

19. The implantable device of claim 10, wherein the wearable device further comprises a decoder configured to wirelessly receive and decode the EMG signal.

20. The implantable device claim 1, wherein the sensor comprises one or more electrodes embedded within the implantable substrate, wherein the sensor comprises two or more electrodes that are spaced equidistantly from a center of each of the two or more electrodes.

21. The implantable device of claim 1, wherein the implantable substrate has a first surface and a second surface on the opposite side of the first surface, and wherein the sensor is embedded within the first surface and the one or more reference electrodes are embedded within the second surface.

22. The implantable device of claim 1, wherein the one or more electrodes comprise sensing electrodes.

23. (canceled)

24. The implantable device of claim 1, wherein the implantable substrate is configured to wrap around a circumference of the one or more muscles of the subject when implanted.

25. The implantable device of claim 1, wherein the implantable substrate is a flat, elongated strip.

26. The implantable device of claim 1, wherein the implantable substrate is flexible and configured to wrap around the one or more muscles of the subject.

27. The implantable device of claim 1, wherein the implantable device further comprises a case connected to a connector of the implantable substrate, the case configured to house one or more electronic components, wherein the case is composed of a biocompatible material and/or a bioinert material.

28. (canceled)

29. An implantable system, comprising:
one or more implantable devices comprising:
an implantable substrate comprising a sensor configured to detect and transmit an electromyography (EMG) signal generated by a muscle of a subject, the implantable substrate being implanted in the subject and comprising a top surface and a bottom surface, both the top surface and the bottom surface being in contact with the one or more muscles of the subject, the sensor being embedded within the top surface and the bottom surface comprising one or more reference electrodes; and
a first processor operatively coupled with the implantable substrate and configured to: i) wirelessly receive the EMG signal from the sensor, and ii) wirelessly transmit the EMG signal; and
one or more wearable devices configured to be attached to the subject, the one or more wearable devices comprising:
a second processor configured to: i) wirelessly receive the EMG signal from the first processor, and ii) wirelessly transmit the EMG signal to a computing device;
a self-contained battery; and
a power transmitter configured to wirelessly transmit energy to the one or more implantable devices via an inductive magnetic field.

30. A method of controlling a prosthesis comprising:
detecting the EMG signal from a subject using the implantable device of claim 1;
wirelessly transmitting the EMG signal from the implantable device to a wearable device;
wirelessly transmitting the EMG signal from the wearable device to an external processing unit;
processing the EMG signal using one or more machine learning classifiers; and
based on the processing, generating a control output for the prosthesis.

31. The method of claim 30, wherein the control output comprises one or more of a continuous joint angle, a movement, or a discrete gesture.

32. The method of claim 30, wherein the one or more machine learning classifiers are one or more of a discrete classifier or a continuous classifier, wherein the discrete classifier comprises a logistic regression classifier.

33. (canceled)

34. (canceled)

35. The method of claim 30, further comprising training the machine learning classifier, wherein training the machine learning classifier comprises:
receiving, via a processor, sensor data comprising one or more of the EMG signal or a motion signal corresponding to a movement of the subject; and
training the machine learning classifier, via the processor, to generate one or more hyperparameters configured to be stored on the processor and configured to control the prosthesis.
